(19) United States
(12) Patent Application Publication
(10) Pub. No.: US 2019/0287659 A1
(43) Pub. Date: Sep. 19, 2019

(54) SYSTEM AND METHOD FOR PROVIDING MODEL-BASED PATIENT ASSIGNMENT TO CARE MANAGERS

(71) Applicant: KONINKLIJKE PHILIPS N.V., Eindhoven (NL)

(72) Inventors: Eran SIMHON, Boston, MA (US); Reza SHARIFI SEDEH, Malden, MA (US)

(21) Appl. No.: 16/287,307

(22) Filed: Feb. 27, 2019

Related U.S. Application Data
(60) Provisional application No. 62/643,451, filed on Mar. 15, 2018.

(51) Int. Cl. G16H 10/60; G16H 50/30; G06N 20/00

(52) U.S. Cl. CPC G16H 10/60; G06N 20/00; G16H 50/30

(57) ABSTRACT

The present disclosure pertains to a system for providing model-based patient assignment to care managers. In some embodiments, the system (i) receives a collection of health information related to a plurality of individuals residing in a predetermined region and known to have similar social determinants of health; (ii) extracts and provides one or more care management-related features of the plurality of individuals and one or more care management activities provided to the individuals to a machine learning model to train the machine learning model; (iii) obtains and provides health information of an individual residing in the predetermined region to the machine learning model to predict an amount of care management time for the individual; (iv) assigns, based on the predicted amount of care management time, the individual to a care manager; and (v) effectuates presentation of a list of assigned individuals to the care manager.

### CROSS-REFERENCE TO PRIOR APPLICATIONS

This application claims the benefit of U.S. Provisional Application No. 62/643,451, filed on 15 Mar. 2018. This application is hereby incorporated by reference herein.

### BACKGROUND

### 1. Field

The present disclosure pertains to a system and method for providing model-based patient assignment to care managers.

### 2. Description of the Related Art

Social behavioral and environmental factors account for more premature deaths than genomics and traditional healthcare-related factors in the United States. In fact, social behavioral and environmental factors account for the majority of all premature deaths in the United States. For example, low socio-economic status may be highly correlated with poor health behavior (e.g., not showing up for appointments, poor adherence to medication, more avoidable emergency department visits, etc.). Care management emphasizes prevention, continuity of care and coordination of care, which advocates for, and links individuals to, services as necessary across providers and settings. Although automated and other computer-assisted care management systems exist, such systems may often fail to properly balance the workloads of care managers, especially given that the actual amount of time spent with patients may be not necessarily correlate with genomics and traditional healthcare-related factors. These and other drawbacks exist.

### SUMMARY

Accordingly, one or more aspects of the present disclosure relate to a system for providing model-based patient assignment to care managers. The system comprises one or more processors configured by machine readable instructions and/or other components. The one or more hardware processors are configured to: receive, from one or more databases, a collection of health information related to a plurality of individuals residing in a predetermined region and known to have similar social determinants of health; extract, from the collection of health information, one or more care management-related features of the plurality of individuals and one or more care management activities provided to the individuals; provide the one or more care management-related features of the plurality of individuals and the one or more care management activities provided to the individuals to a machine learning model to train the machine learning model; obtain health information of an individual residing in the predetermined region; provide, subsequent to the training of the machine learning model, the health information of the individual to the machine learning model to predict an amount of care management time for the individual; assign, based on the predicted amount of care management time, the individual to a care manager, the assignment being determined such that the care manager and other care managers have similar workloads; and effectuate, via a user interface, presentation of a list of assigned individuals to the care manager.

Another aspect of the present disclosure relates to a method for providing model-based patient assignment to care managers with a system. The system comprises one or more processors configured by machine readable instructions and/or other components. The method comprises: receiving, with one or more processors, a collection of health information related to a plurality of individuals residing in a predetermined region and known to have similar social determinants of health from one or more databases; extracting, with the one or more processors, one or more care management-related features of the plurality of individuals and one or more care management activities provided to the individuals from the collection of health information; providing, with the one or more processors, the one or more care management-related features of the plurality of individuals and the one or more care management activities provided to the individuals to a machine learning model to train the machine learning model; obtaining, with the one or more processors, health information of an individual residing in the predetermined region; providing, with the one or more processors, the health information of the individual to the machine learning model subsequent to the training of the machine learning model to predict an amount of care management time for the individual; assigning, with the one or more processors, the individual to a care manager based on the predicted amount of care management time, the assignment being determined such that the care manager and other care managers have similar workloads; and effectuating, via a user interface, presentation of a list of assigned individuals to the care manager.

Still another aspect of present disclosure relates to a system for providing model-based patient assignment to care managers. The system comprises: means for receiving a collection of health information related to a plurality of individuals residing in a predetermined region and known to have similar social determinants of health from one or more databases; means for extracting one or more care management-related features of the plurality of individuals and one or more care management activities provided to the individuals from the collection of health information; means for providing the one or more care management-related features of the plurality of individuals and the one or more care management activities provided to the individuals to a machine learning model to train the machine learning model; means for obtaining health information of an individual residing in the predetermined region; means for providing the health information of the individual to the machine learning model subsequent to the training of the machine learning model to predict an amount of care management time for the individual; means for assigning the individual to a care manager based on the predicted amount of care management time, the assignment being determined such that the care manager and other care managers have similar workloads; and means for effectuating presentation of a list of assigned individuals to the care manager.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based patient assignment to care managers.

FIG. 2 illustrates average amounts of time spent on care management activities, in accordance with one or more embodiments.

FIG. 3 illustrates predicted care management times, in accordance with one or more embodiments.

FIG. 4 illustrates assignment of individuals to care managers, in accordance with one or more embodiments.

FIG. 5 illustrates allocation of individuals to a care manager, in accordance with one or more embodiments.

FIG. 6 illustrates a method for providing model-based patient assignment to care managers, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

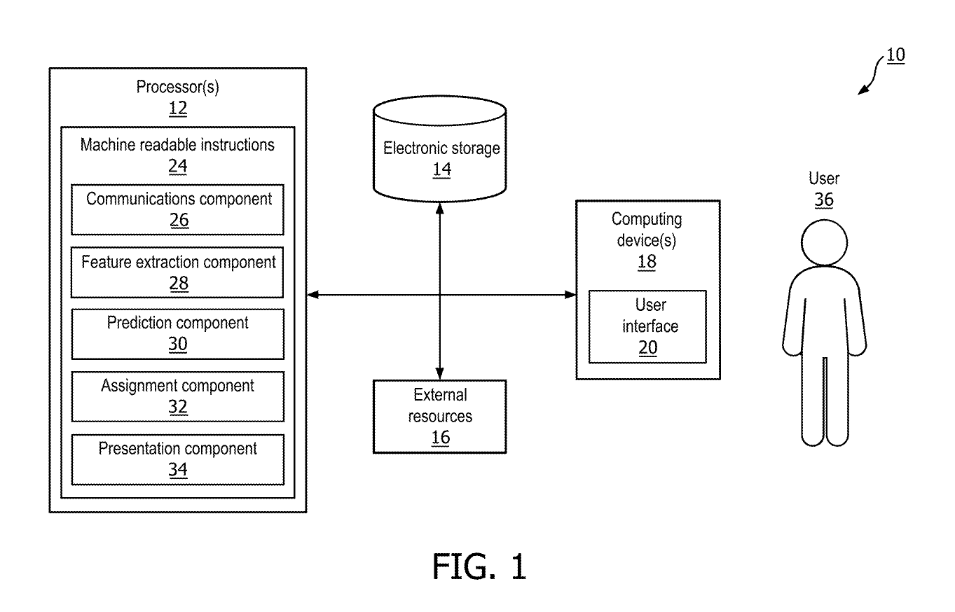

FIG. 1 is a schematic illustration of a system 10 configured for providing model-based patient assignment to care managers. In some embodiments, system 10 is configured to obtain, from a database, a collection of health information including one or more of (i) social determinant information related to a plurality of individuals residing in a predetermined region and known to have similar social determinants of health; (ii) electronic medical records corresponding to the plurality of individuals; (iii) information related to activities that a care manger performed on each of the plurality of individuals annually; and (iv) information related to average or recommended amounts of time to be spent on each care management activity. In some embodiments, system 10 is configured to determine a total amount of time recommended to be (or should be) spent on each of the plurality of individuals by a care manager based on the list of activities associated with each of the plurality of individuals. In some embodiments, system 10 is configured to provide one or more features of the collection of health information and the recommended amounts of time for the care management activities to a machine learning model to train the machine learning model. In some embodiments, system 10 is configured to generate, via the machine learning model, predictions related to an amount of care management time for each of the plurality of individuals. In some embodiments, the prediction corresponds to an amount of time predicted to be spent on each of the plurality of individuals annually. In some embodiments, system 10 is configured to assign each of the plurality of individuals to a care manager based on their corresponding predicted amount of care management time. In some embodiments, the assignment is determined such that a given care manager and other care managers have similar workloads. In some embodiments, system 10 is configured to effectuate presentation of a list of assigned individuals to a corresponding care manager such that the care manager's time is better managed and better service is provided to the individuals.

In some embodiments, system 10 is configured to perform the generation of the amount of care management time prediction or other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, system 10 comprises processors 12, electronic storage 14, external resources 16, computing device 18 (e.g., associated with user 36), or other components.

Electronic storage 14 comprises electronic storage media that electronically stores information (e.g., collection of health information related to a plurality of individuals residing in a predetermined region). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include health information related to the population. In some embodiments, the health information comprises demographic information, vital signs information, medical condition information indicating medical conditions experienced by individuals in the population, treatment information indicating treatments received by the individuals, care management information, and/or other health information. In some embodiments, external resources 16 include sources of information such as databases, websites, etc., external entities participating with system 10 (e.g., a medical records system of a health care provider that stores medical history information of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between user 36 and/or other users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with user 36, and/or other users. In some embodiments, computing device 18 facilitates presentation of a list of individuals assigned to a care manager, or other information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a communications component 26, a feature extraction component 28, a prediction component 30, an assignment component 32, a presentation component 34, or other components. Processor 12 may be configured to execute components 26, 28, 30, 32, or 34 by software; hardware; firmware; some combination of software, hardware, or firmware; or other mechanisms for configuring processing capabilities on processor 12.

It should be appreciated that although components 26, 28, 30, 32, and 34 are illustrated in FIG. 1 as being co-located within a single processing unit, in embodiments in which processor 12 comprises multiple processing units, one or more of components 26, 28, 30, 32, or 34 may be located remotely from the other components. The description of the functionality provided by the different components 26, 28, 30, 32, or 34 described below is for illustrative purposes, and is not intended to be limiting, as any of components 26, 28, 30, 32, or 34 may provide more or less functionality than is described. For example, one or more of components 26, 28, 30, 32, or 34 may be eliminated, and some or all of its functionality may be provided by other components 26, 28, 30, 32, or 34. As another example, processor 12 may be configured to execute one or more additional components that may perform some or all of the functionality attributed below to one of components 26, 28, 30, 32, or 34.

In some embodiment, the present disclosure comprises means for receiving, from one or more databases (e.g., electronic storage 14, external resources 16, etc.), a collection of health information related to a plurality of individuals. In some embodiments, such means for receiving takes the form of communications component 26. As an example, the collection of health information may be related to a plurality of individuals residing in a predetermined region, known to have similar social determinants of health, or having other attributes. In some embodiments, the collection of health information may be representative of 100 or more individuals, 1,000 or more individuals, 10,000 or more individuals, 100,000 or more individuals, 1,000,000 or more individuals, 100,000,000 or more individuals, or other number of individuals. In some embodiments, the collection of health information includes one or more care management-related features of the plurality of individuals. In some embodiments, the care management-related features include one or more of a number of emergency department visits, a number of days hospitalized, a list of chronic diseases, age, marital status, language, a social determinant of health index, or other information. In some embodiments, the plurality of individuals are residing in a predetermined zip code, a plurality of neighboring zip codes, a county, a city, a state, a plurality of neighboring states, a geographic region (e.g., East Coast, West Coast), a country, a plurality of neighboring countries, or other locations. In some embodiments, the predetermined region may be known to include individuals with similar social determinants of health. In some embodiments, social determinants of health include conditions in the environments in which the plurality of individuals are born, live, learn, work, play, worship, and age that affect a wide range of health, functioning, and quality-of-life outcomes and risks. In some embodiments, the conditions include social, economic, and physical aspects in various environments and settings (e.g., school, church, workplace, and neighborhood). In some embodiments, the patterns of social engagement and sense of security and well-being are affected by where the plurality of individuals live. In some embodiments, the social determinants of health have a significant influence on population health outcomes. Examples of these resources include safe and affordable housing, access to education, public safety, availability of healthy foods, local emergency/health services, environments free of life-threatening toxins, or other factors.

In some embodiments, communications component 26 is configured to perform one or more queries based on a predetermined region (e.g., search within a zip code, county, state, etc.), other social determinant parameters, or other criteria to obtain a collection of health information associated with individuals residing in a predetermined region, known to have similar social determinants of health, or having other characteristics. In some embodiments, communications component 26 is configured to obtain a list of available care managers within the predetermined region. In some embodiments, communications component 26 is configured to obtain the collection of health information based on one or more care management-related features of an individual. As such, in some embodiments, the present disclosure comprises means for obtaining health information of an individual residing in a predetermined region. In some embodiments, such means for obtaining takes the form of communications component 26. In some embodiments, communications component 26 is configured to determine, from the health information of the individual, one or more care management-related features of the individual. In some embodiments, the care management-related features include one or more of a number of emergency department visits, a number of days hospitalized, a list of chronic diseases, age, marital status, language, a social determinant of health index, or other information. In some embodiments, communications component 26 is configured to perform one or more queries on one or more databases (e.g., databases stored electronic storage 14, databases available through external resources 16, etc.) based on the care management-related features of the individual to obtain the collection of health information associated with similar individuals having similar care management-related conditions as the individual. In some embodiments, the collection of health information associated with similar individuals indicates care management-related conditions of the similar individuals, one or more care management activities (e.g., follow-up phone calls, arrangement of transportation, assistance with appointment scheduling, etc.) provided to the similar individuals by a care manager, or other information. In some embodiments, communications component 26 is configured to obtain, subsequent to determination of the predicted amount of care management time of the individual (described below), information related to an actual amount of care management time for the individual.

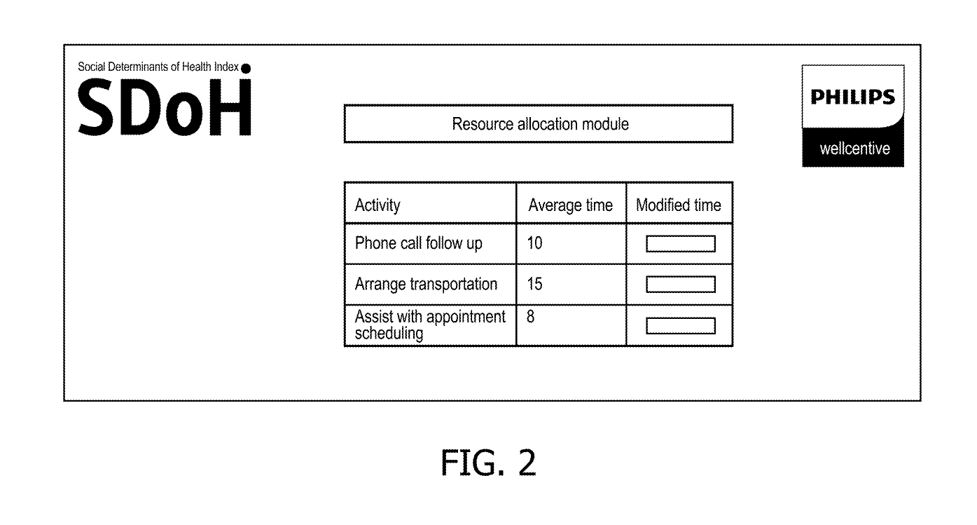

In some embodiments, communications component 26 is configured to obtain, from one or more databases, information related to average or recommended amounts of time to be spent on each care management activity. In some embodiments, responsive to (i) the individual residing in a region with a low social determinant of health index, (ii) care manager availability not meeting individuals' demands, (iii) care manager providing service in a populated region, or (iv) other factors, amounts of time spent on each of care activities may be lower than regions where such conditions are not present. As such, in some embodiments, the average or recommended amounts of time to be spent on each care management activity are determined based on average amounts of time spent per activity for a plurality of populations across one or more regions. In some embodiments, communications component 26 is configured to obtain (e.g., from one or more databases) information related to recommended amounts of time for each of the care management activities provided to the individuals. By way of a non-limiting example, FIG. 2 illustrates average amounts of time spent on care management activities, in accordance with one or more embodiments. In FIG. 2, an individual with a particular set of health conditions may require a phone call follow-up, arrangement of transportation, and assistance with appointment scheduling. As shown in FIG. 2, conducting a follow-up phone call may require (on average) 10 minutes, arranging transportation may require (on average) 15 minutes, and assisting with appointment scheduling may require (on average) 8 minutes. The amounts of time spent on each care management may be determined by determining, per care management activity, average amounts of time spent for performing similar activities across a plurality of populations residing in one or more regions.

In some embodiment, the present disclosure comprises means for extracting one or more care management-related features of the plurality of individuals and one or more care management activities provided to the individuals from the collection of health information. In some embodiments, such means for extracting takes the form of feature extraction component 28. In some embodiments, the care management-related features include one or more of a number of emergency department visits, a number of days hospitalized, a list of chronic diseases, age, marital status, language, a social determinant of health index, or other information. In some embodiments, the care management activities include one or more of identification of individuals who have or may have special needs, assessment of an individual's risk factors, development of a plan of care, referrals and assistance to ensure timely access to providers, coordination of care actively linking the individual to providers, medical services, residential, social, behavioral, and other support services where needed, monitoring, continuity of care, follow-up and documentation, or other activities.

In some embodiment, the present disclosure comprises means for providing the care management-related features of the plurality of individuals and the care management activities provided to the individuals to a machine learning model to train the machine learning model. In some embodiments, such means for providing takes the form of prediction component 30. In some embodiments, prediction component 30 is configured to provide the care management-related features of the similar individuals and the care management activities provided to the similar individuals to the machine learning model to train the machine learning model. In some embodiments, prediction component 30 is configured to provide the recommended amounts of time to the machine learning model to further train the machine learning model. In some embodiment, the present disclosure comprises means for providing the health information of the individual to the machine learning model subsequent to the training of the machine learning model to predict an amount of care management time for the individual. In some embodiments, such means for providing takes the form of prediction component 30. In some embodiments, prediction component 30 is configured to provide, subsequent to the determination of the predicted amount of care management time of the individual (described below), the actual amount of care management time of the individual to the machine learning model to further train the machine learning model.

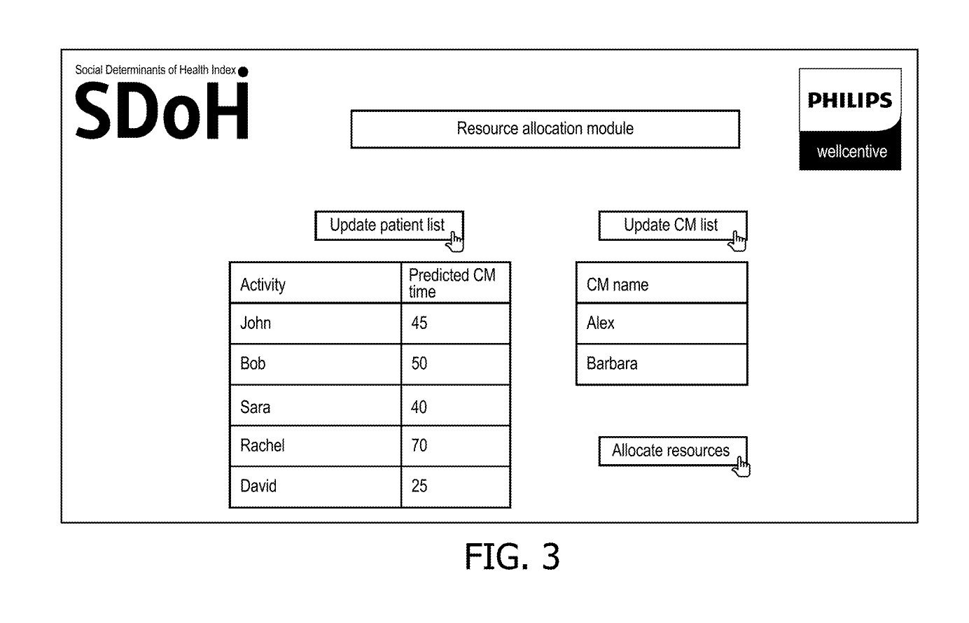

In some embodiments, prediction component 30 is configured to generate predictions related to amounts of care management time for the plurality of individuals via the machine learning model (e.g., as described above). As an example, prediction component 30 may provide, subsequent to the training of the machine learning model, the health information of an individual (or a portion thereof) as input to the machine learning model to cause the machine learning model to output the prediction related to an amount of care management time for the individual (e.g., annual amount of time to be spent on the individual, etc.). In some embodiments, the machine learning model may be trained to output predictions related to types and/or frequencies of care management activities for the individual. In some embodiments, the machine learning model is configured to determine which aspects of the collection of health information, individual's health information, or other information are important. In some embodiments, the machine learning model includes one or more of Linear Regression, Random Forest, Neural Networks, Deep Learning techniques, or other models. By way of a non-limiting example, FIG. 3 illustrates predicted care management times, in accordance with one or more embodiments. As shown in FIG. 3, predicted amounts of time to be spent per individual (e.g., John, Bob, Sara, Rachel, David) for providing care management activities have been determined.

In some embodiments, the machine learning outputs (e.g., the predicted amounts of time) may be modeled (e.g., linear regression, etc.). By way of a non-limiting example, Model 1 illustrates a relationship between one or more care management features and the predicted amount of time to be spent on an individual for providing care management activities, in accordance with one or more embodiments.

CM time=0.2×age−0.1×SDoH index+0.15×number of ED visits Model 1:

Returning to FIG. 1, in some embodiments, the present disclosure comprises means for assigning the individual to a care manager based on the predicted amount of care management time. In some embodiments, such means for assigning takes the form of assignment component 32. In some embodiments, the assignment is further based on the list of available care managers within the predetermined region. In some embodiments, the assignment is determined such that the care manager and other care managers have similar workloads. In some embodiments, assignment component 32 is configured to determine an assignment solution that minimizes the variance of the total workload of care managers as defined by the individuals' predicted amount of care management time. In some embodiments, assignment component 32 is configured to solve an optimization problem as described below to determine the assignments:

Let N be the number of individuals and M be the number of care managers. Let Xᵢⱼ be one if individual i is assigned to care manager j and zero otherwise. Let wᵢ predicted amount of care management time of individual i. The solution of the following optimization problem is the optimal allocation of the individuals to care managers:

$$Min ( variance {\sum }_{i = 1}^{N} {w}_{i} * {x}_{i 1} , {\sum }_{i = 1}^{N} {w}_{i} * {x}_{i 2} , … , {\sum }_{i = 1}^{N} {w}_{i} * {x}_{iM} )$$

$$Subject to {\sum }_{j = 1}^{N} {x}_{ij} = 1 \text{∀} i ; {x}_{ij} ∈ \{ 0 , 1 \} , \text{∀} i , j .$$

As shown in the optimization problem above, the constraints guarantee that each individual is assigned to exactly one care manager. In some embodiments, assignment component 32 is configured to solve the optimization problem via techniques including one or more of LP relaxations, branch and bound, or other techniques.

In some embodiments, assignment component 32 is configured such that assignment of the individual to the care manager is further based on a normalization factor fⱼ. In some embodiments, the normalization factor is determined based on one or more of a number of years of experience of the care manager, a parameter indicative of the care manager's previous year performance, or other factors. For example, responsive to a given care manager being able to deal with a greater workload than the average, the normalization factor corresponding to the given care manager may be greater than one. As another example, responsive to a care manager being able to deal with a smaller workload than the average, the normalization factor corresponding to the care manager may be less than one. As such, assignment component 32 may be configured to solve the below optimization problem to determine the optimal allocation of individuals to care managers:

$$Min ( variance \frac{{\sum }_{i = 1}^{N} {w}_{i} * {x}_{i 1}}{{f}_{1}} , \frac{{\sum }_{i = 1}^{N} {w}_{i} * {x}_{i 2}}{{f}_{2}} , … , \frac{{\sum }_{i = 1}^{N} {w}_{i} * {x}_{iM}}{{f}_{M}} )$$

$$Subject to {\sum }_{j = 1}^{N} {x}_{ij} = 1 \text{∀} i ; {x}_{ij} ∈ \{ 0 , 1 \} , \text{∀} i , j .$$

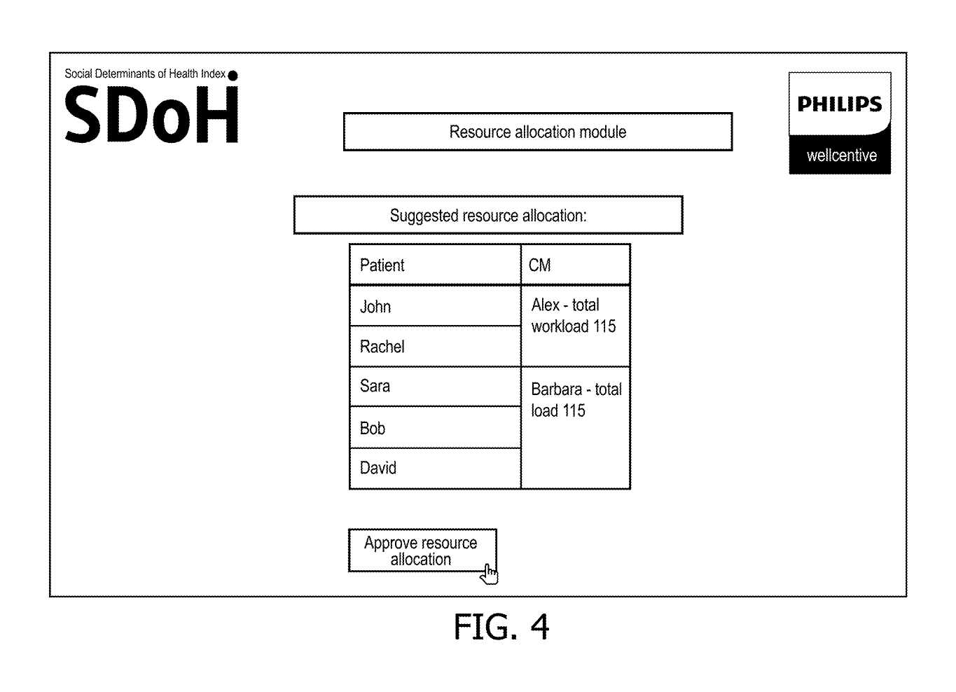

By way of a non-limiting example, FIG. 4 illustrates assignment of individuals to care managers, in accordance with one or more embodiments. As shown in FIG. 4, individuals John and Rachel have been assigned to care manager Alex while individuals Sara, Bob, and David have been assigned to care manager Barbara. In FIG. 4, care manager Barbara is shown to have more individuals assigned to her (3 individuals) compared to care manager Alex (2 individuals); however, based on the predicted amounts of time to be spent per individual (e.g., John, Bob, Sara, Rachel, David) for providing care management activities, the workload for each care manager has been optimized such that the care managers have similar workloads (115 minutes). In some embodiments, assignment component 32 is configured to facilitate approval of the allocations by a care management director (e.g., by providing a prompt on user interface 20).

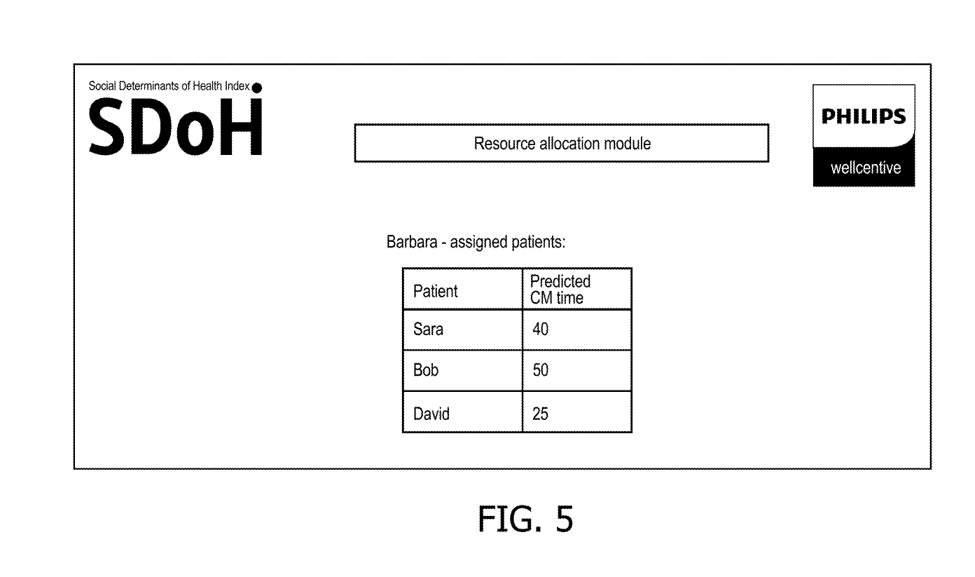

Returning to FIG. 1, in some embodiments, the present disclosure comprises means for effectuating (e.g., via user interface 20) presentation of a list of assigned individuals to user 36 (e.g., the care manager) or other users. In some embodiments, such means for effectuating presentation of the list of assigned individuals takes the form of presentation component 34. In some embodiments, presentation component 34 is configured such that the list further includes the predicted amount of care management time required for each individual. As such, user 36 or other users may be informed, ahead of time, how much time should be spent on providing care management activities to a given individual. In some embodiments, presentation component 34 is configured to effectuate presentation of the list of assigned individuals and their corresponding predicted care management activity types and/or frequencies, such that user 36 (e.g., care manager) or other users may better manage his/her time and provide better service to the individuals. By way of a non-limiting example, FIG. 5 illustrates allocation of individuals to a care manager, in accordance with one or more embodiments. As shown in FIG. 5, presentation component 34 effectuates presentation of a list of assigned individuals (e.g., Sara, Bob, and David) and their corresponding predicted amount of care management time to user 36 (e.g., care manager Barbara).

In some embodiments, presentation component 34 is configured to generate a first interactive element, a second interactive element, or other elements on user interface 20. In some embodiments, presentation component 34 is configured to generate the first interactive element based on a list of individuals that are/to be provided with care management services. In some embodiments, presentation component 34 is configured to generate the second interactive element based on the list of available care managers in the predetermined region. As an example, the first interactive element may correspond to one or more individuals to be assigned to care managers. The second interactive element may correspond to the availability or eligibility of the care managers.

In some embodiments, the first and/or second interactive elements may not be moveable. As an example, these elements may include non-movable textual input fields, icons (e.g., an arrow or +/− signs) on a display, and other interactive elements. In one use case, a user may specify one or more inputs at user interface 20, such as a percent increase or decrease in the normalization factor of the care managers. Additionally, or alternatively, a user may activate (e.g., click or touch) an icon (or button) on user interface 20 to incrementally adjust the normalization factor.

In some embodiments, presentation component 34 is configured to generate the first interactive element on the user interface such that the first interactive element is moveable by a user from a current position of the first interactive element on the user interface to another position on the user interface. In some embodiments, presentation component 34 is configured to generate a second interactive element on the user interface such that the second interactive element is moveable by a user from a current position of the second interactive element on the user interface to another position on the user interface.

As an example, with respect to FIG. 3, the first interactive element may be the predicted amount of care management time corresponding to the patient list, the second interactive element may be the care manager list, and the predicted care management time and/or the care manager list (or portions therein) may be moveable by user 36 (e.g., moveable up or down on the list; removable from the list) or other users. In one scenario, responsive to movement of the first interactive element, the second interactive element, or other interactive elements, the assignment of the individuals to care managers may be adjusted. In another scenario, such adjustments may result in the addition of more care managers, the removal of allocated care managers, and/or the replacement of one care manager with another care manager.

In some embodiments, assignment component 32 may be configured to update the assignment of individuals to care managers responsive to the first interactive element being moved to another position on the user interface, the second interactive element being moved to another position on the user interface, or other user interaction. In some embodiments, prediction component 30 may be configured to update the machine learning model responsive to the first interactive element being moved to another position on the user interface, the second interactive element being moved to another position on the user interface, or other user interaction. As an example, responsive to movement of the first interactive element, the second interactive element, or other interactive elements, the assignment of a group of individuals to a particular care manager may be adjusted. As another example, such adjustments may result in the addition of more care managers (e.g., due to the workload of one or more care managers being unsustainable), the removal of allocated care managers (e.g., the total predicted amount of time for providing care management services does not exceed a care manager's capacity, thus other care manager's assistance may not be required), and/or the replacement of one care manager with another care manager (e.g., a junior care manager may be replaced with a care manager having more experience and efficiency). In one use case, with respect to FIG. 3, a care management director may drag one or more care managers away from the care manager list (e.g., remove one or more care managers from the rotation due to budget cuts, initiation of other social programs in the predetermined region, or other factors affecting socials determinants of health) thus prompting prediction component 30 to update the machine learning model to reflect the changes in the social determinants of health. In this case, for example, prediction component 30 may be configured to generate predictions related to amounts of care management time for the individuals via a machine learning model trained on the changes in the social determinants of health, the new list of available care managers, or other information. In another use case, a care management director may drag an individual's predicted amount of care management time upwards away from the bottom of the patient list to indicate a higher estimated amount of care management time (e.g., due to previous experience with the individual, based on the individual's preferences, etc.). In this case, for example, assignment component 30 may be configured to update the list of assigned individuals to care managers based on the adjusted estimated amount of care management time.

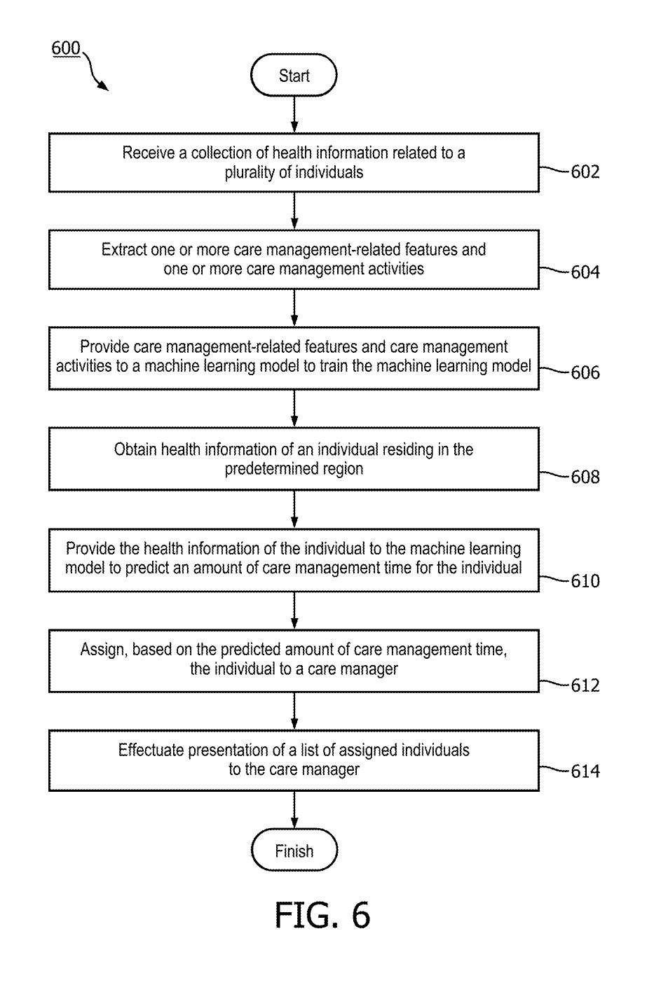

FIG. 6 illustrates a method 600 for providing model-based patient assignment to care managers, in accordance with one or more embodiments. Method 600 may be performed with a system. The system comprises one or more processors, or other components. The processors are configured by machine readable instructions to execute computer program components. The computer program components include a communications component, a feature extraction component, a prediction component, an assignment component, a presentation component, or other components. The operations of method 600 presented below are intended to be illustrative. In some embodiments, method 600 may be accomplished with one or more additional operations not described, or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 600 in response to instructions stored electronically on an electronic storage medium. The processing devices may include one or more devices configured through hardware, firmware, or software to be specifically designed for execution of one or more of the operations of method 600.

At an operation 602, a collection of health information related to a plurality of individuals residing in a predetermined region and known to have similar social determinants of health is received. In some embodiments, operation 602 is performed by a processor component the same as or similar to communications component 26 (shown in FIG. 1 and described herein).

At an operation 604, one or more care management-related features of the plurality of individuals and one or more care management activities provided to the individuals is extracted from the collection of health information. In some embodiments, operation 604 is performed by a processor component the same as or similar to feature extraction component 28 (shown in FIG. 1 and described herein).

At an operation 606, the care management-related features of the plurality of individuals and the care management activities provided to the individuals are provided to a machine learning model to train the machine learning model. In some embodiments, operation 606 is performed by a processor component the same as or similar to prediction component 30 (shown in FIG. 1 and described herein).

At an operation 608, health information of an individual residing in the predetermined region is obtained. In some embodiments, operation 608 is performed by a processor component the same as or similar to communications component 26 (shown in FIG. 1 and described herein).

At an operation 610, the health information of the individual is provided to the machine learning model subsequent to the training of the machine learning model to predict an amount of care management time for the individual. In some embodiments, operation 610 is performed by a processor component the same as or similar to prediction component 30 (shown in FIG. 1 and described herein).

At an operation 612, the individual is assigned to a care manager based on the predicted amount of care management time. In some embodiments, the assignment is determined such that the care manager and other care managers have similar workloads. In some embodiments, operation 612 is performed by a processor component the same as or similar to assignment component 32 (shown in FIG. 1 and described herein).

At an operation 614, a list of assigned individuals to the care manager is presented via a user interface. In some embodiments, operation 614 is performed by a processor component the same as or similar to presentation component 34 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based patient assignment to care managers, the system comprising:
one or more processors configured by machine-readable instructions to:
receive, from one or more databases, a collection of health information related to a plurality of individuals residing in a predetermined region and known to have similar social determinants of health;
extract, from the collection of health information, one or more care management-related features of the plurality of individuals and one or more care management activities provided to the individuals;
provide the one or more care management-related features of the plurality of individuals and the one or more care management activities provided to the individuals to a machine learning model to train the machine learning model;
obtain health information of an individual residing in the predetermined region;
provide, subsequent to the training of the machine learning model, the health information of the individual to the machine learning model to predict an amount of care management time for the individual;
assign, based on the predicted amount of care management time, the individual to a care manager, the assignment being determined such that the care manager and other care managers have similar workloads; and
effectuate, via a user interface, presentation of a list of assigned individuals to the care manager.

2. The system of claim 1, wherein the one or more processors are configured to:
determine, from the health information of the individual, one or more care management-related features of the individual, the one or more care management-related features including one or more of a number of emergency department visits, a number of days hospitalized, a list of chronic diseases, age, marital status, language, or a social determinant of health index;
perform one or more queries based on the one or more care management-related features to obtain the collection of health information associated with similar individuals having similar care management-related conditions as the individual, the collection of health information associated with similar individuals indicating care management-related conditions of the similar individuals and one or more care management activities provided to the similar individuals by a care manager; and
provide the care management-related features of the similar individuals and the one or more care management activities provided to the similar individuals to the machine learning model to train the machine learning model.

3. The system of claim 1, wherein the one or more processors are configured to:
obtain information related to recommended amounts of time for each of the one or more care management activities provided to the individuals;
provide the recommended amounts of time to the machine learning model further train the machine learning model; and
provide, subsequent to the further training of the machine learning model, the health information of the individual to the machine learning model to predict the amount of care management time for the individual.

4. The system of claim 1, wherein the one or more processors are configured to:
obtain, subsequent to the determination of the predicted amount of care management time of the individual, information related to an actual amount of care management time for the individual; and
provide the actual amount of care management time for the individual to the machine learning model to further train the machine learning model.

5. The system of claim 1, wherein assignment of the individual to the care manager is further based on a normalization factor, the normalization factor being determined based on one or both of a number of years of experience of the care manager or a parameter indicative of the care manager's previous year performance.

6. A method for providing model-based patient assignment to care managers, the method comprising:
receiving, with one or more processors, a collection of health information related to a plurality of individuals residing in a predetermined region and known to have similar social determinants of health from one or more databases;
extracting, with the one or more processors, one or more care management-related features of the plurality of individuals and one or more care management activities provided to the individuals from the collection of health information;
providing, with the one or more processors, the one or more care management-related features of the plurality of individuals and the one or more care management activities provided to the individuals to a machine learning model to train the machine learning model;
obtaining, with the one or more processors, health information of an individual residing in the predetermined region;
providing, with the one or more processors, the health information of the individual to the machine learning model subsequent to the training of the machine learning model to predict an amount of care management time for the individual;
assigning, with the one or more processors, the individual to a care manager based on the predicted amount of care management time, the assignment being determined such that the care manager and other care managers have similar workloads; and
effectuating, via a user interface, presentation of a list of assigned individuals to the care manager.

7. The method of claim 6, further comprising:
determining, with the one or more processors, one or more care management-related features of the individual from the health information of the individual, the one or more care management-related features including one or more of a number of emergency department visits, a number of days hospitalized, a list of chronic diseases, age, marital status, language, or a social determinant of health index;
performing, with the one or more processors, one or more queries based on the one or more care management-related features to obtain the collection of health information associated with similar individuals having similar care management-related conditions as the individual, the collection of health information associated with similar individuals indicating care management-related conditions of the similar individuals and one or more care management activities provided to the similar individuals by a care manager; and
providing, with the one or more processors, the care management-related features of the similar individuals and the one or more care management activities provided to the similar individuals to the machine learning model to train the machine learning model.

8. The method of claim 6, further comprising:
obtaining, with the one or more processors, information related to recommended amounts of time for each of the one or more care management activities provided to the individuals;
providing, with the one or more processors, the recommended amounts of time to the machine learning model to further train the machine learning model; and
providing, with the one or more processors, the health information of the individual to the machine learning model subsequent to the further training of the machine learning model to predict the amount of care management time for the individual.

9. The method of claim 6, further comprising:
obtaining, with the one or more processors, information related to an actual amount of care management time for the individual subsequent to the determination of the predicted amount of care management time of the individual; and
providing, with the one or more processors, the actual amount of care management time for the individual to the machine learning model to further train the machine learning model.

10. The method of claim 6, wherein assignment of the individual to the care manager is further based on a normalization factor, the normalization factor being determined based on one or both of a number of years of experience of the care manager or a parameter indicative of the care manager's previous year performance.

11. A system for providing model-based patient assignment to care managers, the system comprising:
means for receiving a collection of health information related to a plurality of individuals residing in a predetermined region and known to have similar social determinants of health from one or more databases;
means for extracting one or more care management-related features of the plurality of individuals and one or more care management activities provided to the individuals from the collection of health information;
means for providing the one or more care management-related features of the plurality of individuals and the one or more care management activities provided to the individuals to a machine learning model to train the machine learning model;
means for obtaining health information of an individual residing in the predetermined region;
means for providing the health information of the individual to the machine learning model subsequent to the training of the machine learning model to predict an amount of care management time for the individual;
means for assigning the individual to a care manager based on the predicted amount of care management time, the assignment being determined such that the care manager and other care managers have similar workloads; and
means for effectuating presentation of a list of assigned individuals to the care manager.

12. The system of claim 11, further comprising:
means for determining one or more care management-related features of the individual from the health information of the individual, the one or more care management-related features including one or more of a number of emergency department visits, a number of days hospitalized, a list of chronic diseases, age, marital status, language, or a social determinant of health index;
means for performing one or more queries based on the one or more care management-related features to obtain the collection of health information associated with similar individuals having similar care management-related conditions as the individual, the collection of health information associated with similar individuals indicating care management-related conditions of the similar individuals and one or more care management activities provided to the similar individuals by a care manager; and
means for providing the care management-related features of the similar individuals and the one or more care management activities provided to the similar individuals to the machine learning model to train the machine learning model.

13. The system of claim 11, further comprising:
means for obtaining information related to recommended amounts of time for each of the one or more care management activities provided to the individuals;
means for providing the recommended amounts of time to the machine learning model to further train the machine learning model; and
means for providing the health information of the individual to the machine learning model subsequent to the further training of the machine learning model to predict the amount of care management time for the individual.

14. The system of claim 11, further comprising:
means for obtaining information related to an actual amount of care management time for the individual subsequent to the determination of the predicted amount of care management time of the individual; and
means for providing the actual amount of care management time for the individual to the machine learning model to further train the machine learning model.

15. The system of claim 11, wherein assignment of the individual to the care manager is further based on a normalization factor, the normalization factor being determined based on one or both of a number of years of experience of the care manager or a parameter indicative of the care manager's previous year performance.
